(19) United States
(12) Patent Application Publication
(10) Pub. No.: US 2010/0136678 A1
(43) Pub. Date: Jun. 3, 2010

(54) METHODS OF DIAGNOSIS OF SPINAL MUSCULAR ATROPHY AND TREATMENTS THEREOF

(75) Inventor: HUNG LI, Taipei (TW)

Correspondence Address: HARNESS, DICKEY & PIERCE, P.L.C., P.O. BOX 828, BLOOMFIELD HILLS, MI, 48303, US

(73) Assignee: Academia Sinica, Taipei (TW)

(21) Appl. No.: 12/704,204

(22) Filed: Feb. 11, 2010

Related U.S. Application Data
(63) Continuation of application No. 11/703,809, filed on Feb. 7, 2007.

(51) Int. Cl. C12N 15/63; C07H 21/02

(52) U.S. Cl. 435/320.1; 536/24.5

(57) ABSTRACT

Methods for treating SMA in a subject comprise administering to the subject a recombinant genetic vector comprising at least one copy of a Stathmin inhibitor. The inhibitor can be a Stathmin expression inhibitor. The inhibitor can be or can encode an antisense or RNAi nucleic acid, such as a siRNA or a shRNA. The copy can be a copy of a host-expressible Stathmin inhibitor or a copy of a host-expressible Stathmin expression inhibitor. Screening methods can comprise the following steps: providing a Stathmin binding agent and a cellular sample from a human subject at least suspected of either having SMA or having a low SMN protein-based condition; contacting the Stathmin binding agent with the cellular sample under conditions in which the agent can specifically bind to Stathmin present in the sample to form complexes, and removing non-specifically bound agent therefrom to leave remaining complexes, and detecting remaining complexes and comparing the level of complexes detected or of the corresponding Stathmin concentration that is present in the sample, to a control level determined from a healthy cellular sample. The detection of an elevated level of complexes or of Stathmin concentration provides a positive screening result.

### CROSS-REFERENCE TO RELATED APPLICATIONS

This application is a continuation of U.S. patent application Ser. No. 11/703,809 filed on Feb. 7, 2007. The entire disclosure of the above application is incorporated herein by reference.

### INTRODUCTION

The present technology relates to methods for treating neurological disorders, particularly spinal muscle atrophy (“SMA”).

The Survival Motor Neuron protein (“SMN”) is thought to be involved in axonal processing. Motor neurons lacking SMN have smaller growth cones and defects in axon outgrowth. However, little is known about these defects and their involvement in spinal muscular atrophy pathogenesis.

SMA is an autosomal recessive disorder characterized by motor neuron degeneration in the anterior horns of the spinal cord, which leads to muscle atrophy and paralysis. The gene responsible for proximal SMA has been identified as homozygous mutations or deletion of the telomeric copy of SMN1 (survival motor neuron 1) gene, (Lefebvre et. al. (1995) Cell 80:155-165). The cetromeric copy of SMN2 is nearly identical to SMN1 but can be distinguished from one another by nucleotide differences in exons 7 and 8. SMN2 can not functionally substitute for SMN1, due to that fact that SMN1 produces over 90% full length mRNA whereas most SMN2 transcripts are deficient in exon7 and encode C terminal truncated SMN protein (Monani et. al. (1999) Hum Mol Genet 8:1177-1183 and Lorson et. al. (1999) Proc Natl Acad Sci 96:6307-6311). The SMN protein is ubiquitously expressed in all tissues and is especially highly expressed in spinal cord motor neurons. SMN levels are correlated with disease severity (Lefebvre et. al., Nat Genet 16:265-9 (1997) and Coovert et al., (1997) Hum Mol Genet 6:1205-14, and demonstrated in SMA mouse models (Hsieh-Li et al. (2000) Nat Genet 24, 66-70 and Monani et al. (2000) Hum Mol Genet 9, 2451-7).

Recent studies suggest SMN may have a critical role in spliceosomal small nuclear ribonucleoprotein (snRNP) biogenesis in all cell types (Liu et al. (1996) Embo J 15, 3555-65; Pellizzoni et al. (1998) Cell 95, 615-24; and Pellizzoni et al. (2002) Science 298, 1775-9). However, the mechanisms by which SMN protein deficiency involved in motor neuron degeneration remain to be elucidated. In the last few years, several articles have been devoted to the study of neuron specific functions of SMN. Motor neurons that lack SMN protein show shorter axons, smaller growth cones, and have defects in axonal mRNA transport and neuromuscular formation, and this suggests SMN protein plays an important role in neuronal cells (Fan et al. (2002) Hum Mol Genet 11, 1605-14; Rossoll et al. (2002) Hum Mol Genet 11, 93-105; Rossoll et al. (2003) J Cell Biol 163, 801-12; Zhang et al. (2003) J Neurosci 23, 6627-37; and McWhorter et al. (2003) J Cell Biol 162, 919-31). Disruption of axonal transport in motor neurons might lead to failure of local protein translation and axon outgrowth; this indicates some mechanisms, such as microtubule assembly, are quite important in axon maintenance.

Microtubules are crucial to the formation of the neuronal cytoskeleton. They function as tracks for neuronal transport, neurite maintenance, as well as the regulation of axonal elongation and growth cone steering. Two categories of protein are involved in the regulation of microtubule dynamics, one stabilizes microtubules and the other destabilizes microtubules. Structural microtubule-associated proteins (MAPs) play roles in promoting microtubule assembly through binding to the lattice of the microtubule polymer, cross linking with microtubules to form bundles, and regulating the interaction between microtubules and actin (Hirokawa, N. (1994) Curr Opin Cell Biol 6, 74-81 and Matus, A. (1988) Annu Rev Neurosci 11, 29-4). Proteins that are involved in microtubule destabilization such as Stathmin and SCG10 have also recently been identified (Sobel, A. (1991) Trends Biochem Sci 16, 301-5; Belmont et al. (1996) Cell 84, 623-31; and Riederer et al. (1997) Proc Natl Acad Sci USA 94, 741-5). Stathmin/Op18 expression is stage-specific in many cell types, being expressed highly in the developing nervous system (Riederer et al (1997) Proc Natl Acad Sci USA 94, 741-5; Schubart, U. K. (1988) J Biol Chem 263, 12156-60; Koppel et al. (1990) J Biol Chem 265, 3703-7; and Amat et al. (1991) Brain Res Dev Brain Res 60, 205-18) and down regulated during terminal differentiation (Amat, et al (1991) Brain Res Dev Brain Res 60, 205-18; Schubar et al (1992) Differentiation 51, 21-32; Amat et al. (1990) Mol Reprod Dev 26, 383-90).

Stathmin promotes microtubule depolymerization by increasing the catastrophes rate of microtubule (Belmont, et al (1996) Cell 84, 623-31; Amat, et al (1991) Brain Res Dev Brain Res 60, 205-18. Stathmin binds tubulin dimers to form a ternary complex and this may cause inhibition of microtubule assembly through a decrease in free tubulin concentration leading to reduced elongation of microtubule (Belmont, L. D. & Mitchison, T. J. (1996) Cell 84, 623-31).

### SUMMARY

Accordingly, the present disclosure provides methods for treating SMA in a subject comprising administering to the subject a recombinant genetic vector comprising at least one copy of a Stathmin inhibitor. The inhibitor can be a Stathmin expression inhibitor. In some embodiments the inhibitor is or can encode an antisense or RNAi nucleic acid, such as a siRNA or a shRNA. The copy can be a copy of a host-expressible Stathmin inhibitor or a copy of a host-expressible Stathmin expression inhibitor.

In some embodiments, nucleic acid vectors are useful herein to increase or decrease Stathmin transcription/expression levels and/or to introduce nucleic acids encoding enhanced Stathmin proteins such as constitutively active Stathmin proteins or those that can knock-down Stathmin expression levels in the target cell.

The present disclosure provides screening methods for use in diagnosis of SMA or a low-SMN protein-based condition. In some embodiments, a method comprises the following steps: providing a Stathmin binding agent and a cellular sample from a human subject at least suspected of either having SMA or having a low SMN protein-based condition; contacting the Stathmin binding agent with the cellular sample under conditions in which the agent can specifically bind to Stathmin present in the sample to form complexes, and removing non-specifically bound agent therefrom to leave remaining complexes, and detecting remaining complexes and comparing the level of complexes detected or of the corresponding Stathmin concentration that is present in the sample, to a control level determined from a healthy cellular sample. The detection of an elevated level of complexes or of Stathmin concentration provides a positive screening result. In some embodiments the Stathmin binding agent can be an anti-Stathmin antibody or a detectably labeled Stathmin binding agent. In some embodiments, the screening result is combined with at least one further test result in order to provide a diagnosis of SMA or a low-SMN protein-based condition. Further test results may include any one of a result of a test for functional SMN protein expression level, a result of a test for nuclear gem occurrence level, a result of a test for snRNP formation levels, or a combination thereof.

The present disclosure provides kits for use within any of the diagnostic or screening methods described herein. In some embodiments, kits can be used to determine the presence, such as for example, high, normal or level expression levels, or the absence of Stathmin in a test sample.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

The patent or application file contains at least one drawing executed in color. Copies of this patent or patent application publication with color drawing(s) will be provided by the USPTO upon request and payment of the necessary fee.

FIG. 1A illustrates a Western blot analysis of SMN expression of inducible-knockdown SMN NSC34 cells showing SMN levels decreasing after 48 hr transient transfection;

FIG. 1B illustrates an immunocytochemical analysis confirming SMN is knocked down in some NSC34 cells (indicated by the white circle) 48 hr after transient transfection. Cells are stained with SMN antibody (red). 4′6-Diamidino-2-pheylindole (DAPI) is used to stain nuclei (blue). (Scale bar: 10 μm.);

FIG. 1C in the upper panel illustrates stable cell lines carrying SMN-specific shRNA are induced with 1, 2 μg/ml dox. Whole-cell extracts are prepared after 96 hr and SMN expression is analyzed by Western blot. The lower panel illustrates stable cells carrying shRNA for SMN are induced with 1 μg dox and are analyzed for SMN levels at indicated time points. α-Tubulin in A, C is used as a loading control;

FIG. 2A illustrates Stathmin that is differentially expressed in the spinal cords of SMA-like mice and in knockdown SMN NSC34 cells with a comparison of magnified gel areas. The red circle indicates that Stathmin is differentially expressed between control (left panel) and knockdown SMN NSC34 cells (right panel);

FIG. 2B illustrates stable cell lines that carry SMN-specific shRNA that are not induced or induced with 1 μg/ml dox for 72 h or 96 h. Stathmin and α-Tubulin (control) expression are detected by Western blot;

FIG. 2C illustrates spinal cord proteins that are extracted from four Type I SMA-like mice and their control littermates, and are analyzed by Western blot. Stathmin and β-Actin (control) signals are quantified separately. The Stathmin levels are calculated by normalizing the Stathmin signal with β-actin;

FIG. 2D illustrates cerebellum proteins that are extracted from four Type I SMA-like mice and their control littermates, and are analyzed by Western blot. Stathmin and β-Actin (control) signals are quantified separately;

FIG. 3A illustrates transverse sections of spinal cords form Type I SMA-like mice (upper panel) and control littermates (lower panel) that are double stained for Stathmin and ChAT. Stathmin signals increase in motor neuron cell bodies of SMA-like mice (as showed by arrows);

FIG. 3B illustrates quantification of accumulated Stathmin in motor neurons for four pairs of spinal cords from SMA-like mice and control littermates. The number of both ChAT and ChAT/Stathmin positive signal motor neurons from spinal cords are calculated. P=0.012 when compared Stathmin positive motor neurons in control mice with SMA-like mice by t-test;

FIG. 3C in the upper panel illustrates Stathmin levels that are in inducible-knockdown SMN NSC34 cells without (left panel) or with dox treatment (right panel) by immunocytochemical analysis. The lower panel illustrates intensities of Stathmin in control and knockdown SMN NSC34 cells that are captured by LSM510 and are counted by MetaMorf software. *P<0.01(Scale bars: 20 μm.);

FIG. 4A illustrates immunoflurorescent labeling of transiently transfected NSC34 cells with Flag-tagged Stathmin for 48 h. Stathmin is stained with Flag antibody (red) and microtubule structure is stained with α-Tubulin antibody (green). The right panel is high magnification of the boxed white circle in the left panel. DAPI is used to stain nuclei (blue);

FIG. 4B illustrates expression of flag-tagged Stathmin in NSC34 cells that are immunostained for F-actin (green), α-Tubulin (red), flag (blue), and DAPI (White);

FIG. 4C illustrates control in the upper panel, and knockdown SMN cells in the lower panel that are immunostained for α-tubulin (red), Neurofilament H (green), and DAPI (blue). (Scale bars: 10 μm);

FIG. 5A illustrates a statistical analysis of Stathmin expression levels in spinal cords from SMA-like mice compare with control littermates at embryonal day 12.5, 15.5 and 19.5 by t-test. (n=four to five), *, p<0.05 (versus control), **, p<0.01 (versus control);

FIG. 5B illustrates E18.5 and 7 postnatal day whole-mount diaphragm muscles from SMA-like embryo/mouse(upper panel) and their control littermates (lower panel) are stained with Neurofilament H (green) and α-bungarotoxin (red) (Scale bars: 50 μm.);

FIG. 6A illustrates control eGFP vector that are transfected into wild-type E13.5 primary cultured motor neurons;

FIG. 6B illustrates Stathmin-eGFP (green) is transiently transfected into wild-type E13.5 motor neuron. Axon processes are stained with βIII-Tubulin (Red) and motor neurons are characterized by ChAT staining (Blue);

FIG. 6C illustrates a magnified large view of Stathmin expression causing a fragmented axon process (Scale bars: 10 μm.);

FIG. 7A illustrates a Western blot of subcellular fractionation of spinal cord lysates from Type I or Type II SMA-like mice and their control littermates (n=five to eight per group) probed for Stathmin. β-Actin and Syntaxin-1 are used as a loading control;

FIG. 7B illustrates expression of flag-tagged Stathmin in NSC34 cells are immunostained for flag (green), golgi apparatus (red), and DAPI (blue);

FIG. 7C illustrates transverse sections of spinal cords from Type I SMA-like mice and control littermates are triple stained for Stathmin (red), Caspase 2 (green), and ChAT (light blue). Caspase 2 signals increase in Stathmin-positive motor neurons of SMA-like mice (indicated by arrows). DAPI is used for nucleus staining;

FIG. 7D illustrated NSC34 cells treated with H₂O, 0.4 μg/ml Ariamycin, 25 μg/ml 5′-Fluorouracil or 2 mg/ml puromycin for 24 hours or 48 hours;

FIG. 8A illustrates spinal motor neurons extend long axons during embryonic stage, axons migrate to muscle end-plate and form neuromuscular junctions;

FIG. 8B illustrates a portion of the motor neurons in SMA that remain as extended long axons during embryonic stage, axons migrate to muscle end-plate and form neuromuscular junctions; and

FIG. 8C illustrates that during the postnatal period, Stathmin over-expression causing motor axon degeneration.

It should be noted that the figures set forth herein are intended to exemplify the general characteristics of an apparatus, materials and methods among those of this technology, for the purpose of the description of such embodiments herein. These figures may not precisely reflect the characteristics of any given embodiment, and are not necessarily intended to define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific inventions claimed in this application or in such other applications as may be filed claiming priority to this application, or patents issuing therefrom. The following definitions and non-limiting guidelines must be considered in reviewing the description of the technology set forth herein. In particular, although the present disclosure will be discussed in some embodiments as relating to methods of diagnosis of spinal muscular atrophy and treatments thereof, such discussion should not be regarded as limiting the present disclosure to only such applications.

The headings (such as “Introduction” and “Summary”) and sub-headings used herein are intended only for general organization of topics within the present disclosure, and are not intended to limit the disclosure of the technology or any aspect thereof In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. All references cited in the “Detailed Description” section of this specification are hereby incorporated by reference in their entirety, for all purposes. In the event that one or more of the incorporated references, literature, and similar materials differs from or contradicts this application, including but not limited to defined terms, term usage, described techniques, or the like, this application controls.

The description and specific examples, while indicating some embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given some embodiments of this technology have, or have not, been made or tested.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, and methods of this technology.

The present technology provides methods for treating SMA in mammals, methods for diagnosing SMA in mammals, and kits for screening for SMA in mammals. In some embodiments, a mammal is a human. In some embodiments, methods for treating SMA comprise administering a recombinant genetic vector comprising at least one copy of a Stathmin inhibitor.

Stathmin is a highly conserved protein of about 17 kDa (see SEQ ID NO. 3). Its function as an important regulatory protein of microtubule dynamics has been well characterized. Microtubules are highly dynamic structures that continuously alternate between assembly and disassembly. Stathmin performs an important function in regulating rapid microtubule remodeling of the cytoskeleton in response to the cell's needs. Microtubules are cylindrical polymers of α,β-tubulin and microtuble assembly is in part determined by the concentration of free tubulin in the cytoplasm. At low concentrations of free tubulin, the growth rate at the microtubule ends is slowed and results in an increased rate of depolymerization also known as disassembly.

Stathmin interacts with two molecules of dimeric α,β-tubulin to form a tight ternary complex called the T2S complex. One mole of Stathmin binds to two moles of tubulin dimers through the Stathmin-like domain (SLD). When Stathmin sequesters tubulin into the T2S complex, tubulin becomes nonpolymerizable. Without tubulin polymerization, there is no microtubule assembly. Through this mechanism, Stathmin promotes microtubule disassembly without acting directly on the microtubule ends.

The rate of microtubule assembly is an important aspect of cell growth therefore associating regulation of Stathmin with cell cycle progress. Regulation of Stathmin is cell cycle dependent and controlled by the cell's protein kinases in response to specific cell signals. Phosphorylation at four serine residues on Stathmin named Ser16, Ser25, Ser38 and Ser63 causes weakened Stathmin-tubulin binding. Stathmin phosphorylation increases the concentration of tubulin available in the cytoplasm for microtubule assembly. For cells to assemble the mitotic spindle necessary for initiation of the mitotic phase of the cell cycle, Stathmin phosphorylation must occur. Without microtuble growth and assembly, the mitotic spindle cannot form, and the cell cycle is arrested. At cytokinesis, the last phase of the cell cycle, rapid dephosphorylation of Stathmin occurs to block the cell from entering back into the cell cycle until it is ready.

According to the present technology, high, or over expression levels of Stathmin in a cell cause motor neuron death in the cell. In addition, increased, high, or over expression levels of Stathmin is an indication of SMA. The expression level of Stathmin can be used as a biomarker for screening for SMA. Furthermore, knock down SMN levels of expression correlates with the up-regulation of Stathmin. In animal models, the up-regulation of Stathmin in SMA-like mice damages microtubule maintenance during axon outgrowth, causing axon degeneration, and contributing to axonopathy found in the diaphragm muscles of SMA-like mice. In addition, increased Stathmin accumulates in spinal cord motor neurons of SMA-like mice than control mice.

According to the present disclosure, using proteomic analysis a microtubule destabilizing factor, Stathmin, is up regulated in SMN-knockdown NSC34 cells. Over-expression of Stathmin disrupts microtubule organization in NSC34 cells. In addition, Stathmin over-expressed in primary cultured motor neurons results a dying-back axonal defect and highly expressed Stathmin causes detrimental injury in axon extension. Furthermore, Stathmin accumulation in motor neurons causes golgi fragmentation and motor neuron death. Accordingly, over-expression of Stathmin causes internal and external damage to motor neurons, therefore, Stathmin is involved in SMA pathogenesis. Furthermore, defects in motor neuron axon extension correlate with microtubule destabilization.

In some embodiments, a method for treating SMA in a subject comprises administering to the subject a recombinant genetic vector comprising at least one copy of a Stathmin inhibitor. The inhibitor can be a Stathmin expression inhibitor. In some embodiments the inhibitor is or can encode an antisense or RNAi nucleic acid, such as a siRNA or a shRNA. The copy can be a copy of a host-expressible Stathmin inhibitor or a copy of a host-expressible Stathmin expression inhibitor. In some embodiments, the target sequence of the antisense or RNAi nucleic acid is CGT TTG CGA GAG AAG GAT A (SEQ ID. NO. 6).

In some embodiments, nucleic acid vectors are useful herein to increase or decrease Stathmin transcription/expression levels and/or to introduce nucleic acids encoding enhanced Stathmin proteins such as constitutively active Stathmin proteins or those that can knock-down Stathmin expression levels in the target cell.

Knockdown refers to introduction into a cell of a nucleobase polymer, such as a nucleic acid, that can decrease the level of expression of a selected target gene; this differs from knock-out or gene silencing techniques that would eliminate target gene expression altogether. Stathmin knockdown can be performed by use of RNAi technology, such as by introducing, into the cell, either (1) a controlled amount of Stathmin RNA-targeted siRNA or morpholino oligo molecules, or (2) a host-cell-expressible construct encoding Stathmin RNA-targeted shRNA. In some embodiments, nucleic acid from which a Stathmin RNA-targeted shRNA can be expressed, can be used for this purpose. For example, MISSION shRNA nucleic acids (knockdown RNAi nucleic acids available from Sigma-Aldrich, Inc., St. Louis, Mo., USA) can be used, according to manufacturer's instructions. The expressible, shRNA-encoding sequence is operably attached to a promoter, e.g., a U6 promoter. The resulting construct is delivered to the cell for nuclear importation and expression.

Sequences useful for preparing Stathmin knockdown RNAi nucleic acids can be readily obtained from, e.g., SEQ ID NOs: 1 and 2 hereof, and can be prepared according to methods known in the art, such as those described in for example Ui-Tei et al., (2004) Nucl. Acids Res. 32:936-48. The shRNA sequences identified can then be included in constructs and delivered, e.g., via vectors, to motor neuron cells. One such method for Stathmin knockdown is described in for example Holmfeldt et al., (2006) Mol. Biol. Cell. 17(7):2921-2930, which employs an Epstein-Barr viral vector for constitutive expression of Stathmin-targeted shRNA.

Commercially available Stathmin-targeted shRNA nucleic acids, or the sequences thereof, can be used. Examples of these include SURESILENCING shRNA STMN1 LAP18/Lag Human Stathmin 1/oncoprotein 18 (Stathmin-targeting shRNA available from SuperArray Bioscience Corporation, Frederick, Md., USA), and HuSH 29 mer shRNA Constructs against STMN1 (Cat. No. TR318815, available from OriGene Technologies, Inc., Rockville, Md., USA).

Other approaches for control of Stathmin expression that have been developed can be employed in place of a Stathmin knockdown approach hereof. For example, in some embodiments, a Stathmin anti-sense or siRNA-based Stathmin gene silencing approach can be used. See, for example, Alli et al., (Aug. 14, 2006) Oncogene [Epub ahead of print]. Alternatively, a Stathmin RNA-degrading activity, such as an anti-Stathmin ribozyme, can be expressed from a recombinant construct introduced into a target cell. See, for example Mistry et al., (2001) Antisense Nucl. Acid Drug Dev. 11(1):41-9.

A vector can be used to deliver the nucleic acid construct(s) to motor neuron cells. In some embodiments, the vector can be a recombinant viral vector, containing either a full or partial complement of viral chromosomal nucleic acid. See, for example, Federici et al. (2006) Muscle & Nerve 33(3):302 (2006). In the case of virulent viruses for use as, or in forming, viral vectors, these can contain a partial complement of viral chromosomal nucleic acid and can be non-virulent, in some embodiments hereof. Among useful viruses for forming recombinant viral vectors are adenoviruses (AV), adeno-associated viruses (AAV), herpes viruses, and lentiviruses; and in recombinant adenoviral, herpes viral, and lentiviral vectors, these can be non-virulent. See, for example, Haase et al., (1997) Nature Med. 3:429-436; Vincent et al., (2004) Neuromolec. Med. 6(2-3):79-85; Latchman (1999) J. R. Soc. Med. 92(10:566-570; and Wong et al., (2006) Hum. Gene Ther. 17(1):1-9.

In some embodiments, exemplary viruses for use in preparing a viral vector can be: a first or second generation adenovirus or an Epstein-Barr virus or herpes simplex virus. In some embodiments, an AAV or a second generation AV can be used to form a recombinant viral vector hereof. See, for example, Barton et al., (2006) Molec. Ther. 13:347-56; and Alba et al., (2005) Gene Ther. 12 Suppl.(1):S18-27 (October 2005).

Such genetic vectors can be administered once or more than once in a course of treatment, and the same vector can be administered each time or a different vector can be used. Such vectors can be administered in conjunction with a further, non-genetic-vector therapeutic agent, whether a pharmaceutical, nutraceutical, or other medically acceptable beneficial substance. In some embodiments, such further agent(s) can be any one or more of substances that directly or indirectly inhibit Stathmin gene transcription, Stathmin gene transcript processing/splicing, Stathmin expression, or Stathmin activity.

The present technology provides screening methods for use in diagnosis of SMA or a low-SMN protein-based condition. In some embodiments, a method comprises the following steps: providing a Stathmin binding agent and a cellular sample from a human subject at least suspected of either having SMA or having a low SMN protein-based condition; contacting the Stathmin binding agent with the cellular sample under conditions in which the agent can specifically bind to Stathmin present in the sample to form complexes, and removing non-specifically bound agent therefrom to leave remaining complexes, and detecting remaining complexes and comparing the level of complexes detected or of the corresponding Stathmin concentration that is present in the sample, to a control level determined from a healthy cellular sample. The detection of an elevated level of complexes or of Stathmin concentration provides a positive screening result. In some embodiments the Stathmin binding agent can be an anti-Stathmin antibody or a detectably labeled Stathmin binding agent. In some embodiments, the screening result is combined with at least one further test result in order to provide a diagnosis of SMA or a low-SMN protein-based condition. Further test results may include any one of a result of a test for functional SMN protein expression level, a result of a test for nuclear gem occurrence level, a result of a test for snRNP formation levels, or a combination thereof.

The present disclosure provides kits for use within any of the diagnostic or screening methods described herein. In some embodiments, kits can be used to determine the presence, such as for example, high, normal or level expression levels, or the absence of Stathmin in a test sample. The test sample can be from a test subject or patient that may have SMA. In some embodiments, the test sample can be from an embryo for a determination of a likelihood of SMA in a mammal after birth. Such kits typically comprise two or more components necessary for performing a diagnostic assay. In some embodiments, components may be compounds, reagents, containers and/or equipment. For example, one container within a kit may contain an antibody or fragment thereof that specifically binds to Stathmin. Such antibodies or fragments may be provided attached to a support material. One or more additional containers may enclose elements, such as reagents or buffers, to be used in the assay. In some embodiments, kits may contain a detection reagent as described herein that contains a reporter group or label suitable for direct or indirect detection of antibody binding.

In some embodiments, kits can comprise instructions for use thereof to screen for an elevated Stathmin concentration. Kits can comprise instructions for combining a screening result with at least one further test or screening result to provide a diagnosis of a SMA or a low-SMN protein based condition. In some embodiments, the further test or screening result includes any one of a result of a test for functional SMN protein expression level, a result of a test for nuclear gem occurrence level, a result of a test for snRNP formation levels, or a combination thereof Kits can be used to screen for SMA in a test subject that is a mammal, such as a human.

### Examples

Aspects of present disclosure may be further understood in light of the following examples, which should not be construed as limiting the scope of the present disclosure in any way.

### Materials and Methods

Constructs: SMN shRNA plasmid is constructed as follows. Two chemically synthesized oligonucleotides, GATCCCGTAGCTAATAGTACAGAACTTCAAGAGAGTTCTGTACTATTAGCTAC TTTTTT (SEQ. ID NO. 4) and ′AGCTTAAAAAAGTAGCTAATAGTACAGAACTCTCTTGAAGTTCTGTACTATT AGCCAT (SEQ ID NO. 5), are annealed and are inserted at the BglII site and HindIII site of pSuperior.puro vector (OligoEngine), which is then designated pSuperior-SMN shRNA. EGFP-epitope tagged expression plasmid pEGFP-Stathmin is generated by cloning full-length Stathmin cDNA (SEQ ID. NO.1) in-frame into the BamHI sites of the pEGFP-C3 vector (BD Biosciences). The 0.45-kilobase fragment of Stathmin coding region is cloned into EcoRI site of the pCMV2-Flag vector (Sigma).

Cell culture, Transfection, and Stable clone selection: The cell line NSC34 is routinely cultured in Dulbecco's modified Eagle's medium (DMEM) supplemented with 10% heat-inactivated FBS, 1 mM glutamine, and 100 IU/ml penicillin and 100 μg/ml streptomycin (Life Technologies) and is incubated at 37° C. in a 5% CO₂ humidified atmosphere. Transfection is performed using LipofectAMINE™2000 (Invitrogen) according to the manufacturer's recommendations. To generate inducible RNAi-stable clones, NSC34 cells are first transfected with pcDNA6/TR vector (Invitrogen) and individual Blasticidin (Invitrogen) resistant clones are selected. Next, the NSC34-TR cell line is transfected with pSuperior-SMN shRNA and selected single clones by adding 1 μg/ml puromycin. Finally, Western blot experiments are used to evaluate SMN expression levels in each stable clone with or without doxycycline (Dox, Sigma) treatment.

Isolation and primary culture of motor neurons is followed as described in Wiese et al. (1999) Eur J Neurosci 11, 1668-76; and Duong et al. (1999) Br J Pharmacol 128, 1385-92. For primary motor neuron transfection, 0.1M polyethylenimine (PEI, Sigma) reagent is used as described in Scherer et al, (2002) J Gene Med 4, 634-43. Briefly, Opti-MEM (Gibco) diluted plasmid DNA (Stathmin-eGFP or eGFP) is added to Opti-MEM diluted polyethylenimine solution (0.1M PEI, in 5% glucose) in equal volumes while vortexing (giving rise to an N/P ratio of 10). After 15 incubation, the mixture is added into the culture medium. After 36 hours transfection, cells are harvested for immunocytochemical analysis.

Proteomics analysis: Spinal cords from SMA-like mice or control mice having Knockdown SMN NSC34 Cells or control cells are lysed with lysis buffer (150 mM NaCl, 50 mM Tris/HCl pH8.0, 2 mM EDTA, 0.5% sodium deoxycholate, 1% Nonidet P-40, 1 mM Na₃VO₄, 50 mM NaF) supplemented with protease inhibitor cocktail (Rhoche). Then, the cell lysates are centrifuged for 20 min at 12,000 rpm using a refrigerated centrifuge, and the supernatants are collected and precipitated by addition of 10% trichloroacetic acid (TCA) in acetone. Protein pellets are centrifuged and ice-cold acetone used to wash the pellets several times. Finally, the protein is dissolved in 350 μl of a rehydration solution containing 8M urea, 2% CHAPS, 0.5% ampholyte pH 4-7, and 20 mM DTT. It is then vortexed and left at room temperature overnight. The rehydration solution is centrifuged at 12,000 rpm for 20 min and the protein concentration of the supernatant is adjusted to approximately 500 μg/350 μl.

Isoelectric focusing (IEF) is performed using ready-to-use Immobiline Dry-Strips, linear pH gradient 4-7, length 18 cm (Amersham Biosciences), and is run on IPGphor electrophoresis unit (Amersham Biosciences). Strips are rehydrated in the presence of rehydration solution under constant low voltage (30 V) for 20 h. The first-dimensional IEF isis conducted at 18° C., programmed with voltages of 500 V for 0.5 h, 1000 V for 0.5 h, 1500 V for 0.5 h, 2000 V for 0.5 h, 3000 V for 0.5 h, 6000 V for 1 h, and 8000 V for 80 kVh. After the first-dimensional IEF, the strips are incubated for 15 min in an equilibration buffer, containing 50 mM Tris-HCl, pH 8.8, 6 M urea, 30% v/v glycerol, 2% w/v SDS and dithioerythritol (DTE). This is followed by incubation in equilibration buffer plus iodoacetamide for 15 min. The second-dimensional electrophoresis using Protean II xi Multi-Cells (Bio-Rad Laboratories, Hercules, Calif., USA) is performed on 8-18% gradient polyacrylamide gels at 10 mA for 1 h and then at 35 mA until the buffer front line is 5-10 mm from the bottom of the gel. Proteins are stained with Sypro Ruby. The electrophoretic patterns of 2-D gels are compared and analyzed using PDQUEST software, Version 6.2.1 (Bio-Rad Laboratories). For comparisons of proteins among gels, normalization is conducted against the total intensity of all spots present in the gel. The relative spot intensities are then subjected to statistical analysis by applying the nonparametric test of Mann and Whitney. P values less than 0.5 are considered statistically significant. For mass spectrometry, the spots of interest are excised from the gels and sequentially are washed for 1 hour in 50 mM ammonium bicarbonate and in 50% acetonitrile before gel digestion. After washing, gel pieces are processed in acetonitrile, dried, and then are rehydrated with 50 mM ammonium bicarbonate containing 0.01% of sequence grade trypsin (Promega, Madison, Wis.). After an overnight digestion at room temperature, the peptides are extracted twice with 50% acetonitrile and 0.1% trifluoroacetic acid and are vortexed and are sonicated (each for 20 minutes). After drying, the peptide mixtures are resuspended in 10 ul of 50% acetonitrile and 5% TFA and are sonicated for 30 seconds for direct spotting on a MALDI and API-TOF analysis. Mass spectrometry analysis can be carried out at the core facilities of the Proteomics and Structural Biology Research Center (Academia Sinica, Taipei, Taiwan).

Western Blot: All Western blots are performed as in described Hsieh-Li et al. (2000), Nat Genet 24:66-70. The membranes are incubated with antibodies direct against SMN (1:10,000, BD Transduction Laboratories), Stathmin (1:10,000, Calbiochem), α-Tubulin (1:30,000, Amersham Pharmacia Biotech), β-Actin (1:10,000, Sigma), Calnexin (1:500, Chemicon) and cleavage-caspase 3 (1:1000, Cell Signaling). Western blot quantification is performed by scanning the autoradiographs with a computerized densitometer. Signal intensities are determined by densitometry analysis (Fuji film LAS-1000 plus pictography) using the program Phoreticx 1D (Phoreticx International).

Immunocytochemistry: NSC34 cells or primary motor neurons grown on glass coverslips are fixed in 4% paraformaldehyde, permeabilized with 0.5% Triton X-100 in PBS, and blocked with 3% BSA in PBS. The primary antibodies against SMN, ChAT (Chemicon), GOLPH (Golgi marker, Abeam), Neurofilament H (Chemicon), α-Tubulin, βIII-Tubulin (Chemicon), flag (Sigma), and Stathmin (Calbiochem) are added (diluted to 1:500) and then the appropriate fluorescence dye conjugated secondary antibodies (Molecular probe, Invitrogen) are added. Finally, samples are treated with DAPI (Sigma) for 5 min to identify nuclei and mounted with fluorescence mounting solution (DAKO). Confocal images are obtained using an LSM510 Meta confocal laserscanning microscope (Zeiss, Oberkochen, Germany). The LSM5 Image Browser software is used for image acquisition.

Immunohistochemistry (IHC): The SMA mouse model that can be used in a study can be generated as described in Hsieh-li et al. (2000), Nat Genet 24:66-70 and in commonly assigned U.S. Pat. No. 6,245,963 to Li et al., issued Jun. 12, 2001. Spinal cords cryosections of 8 μm thickness are incubated with the primary antibodies against Stathmin (1:400, Calbiochem), ChAT (1:200, Chemicon), and Caspase 2 (1:100, Chemicon), and then are incubated with the appropriate secondary antibodies conjugated with a fluorescence dye (Molecular probe, Invitrogen). Finally, samples are incubated with DAPI (Sigma) for 5 min to identify nuclei and mounted with fluorescence mounting solution (DAKO). Negative controls are included within the cells incubated with IgG of the same species as the primary antibodies instead of the primary specific antibodies. Confocal images are obtained using an LSM510 Meta confocal laserscanning microscope (Zeiss, Oberkochen, Germany). The LSM5 Image Browser software is used for image acquisition.

Whole-mount neurofilament/α-bungarotoxin staining: For whole mount immunostaining, embryos at embryonic stage 18.5 (E18.5), and SMA-like mice with control littermates diaphragm muscles are dissected out. Tails from each embryo are cut for genotyping as described Hsieholi et al. (2000), Nat Genet 24:66-70. Immunostaining of diaphragm muscles is performed as described in Lin et al. (2005) Neuron 46:569-79. Conforcal micrographies are captured on a Zeiss LSM5 PASCAL microscope.

Subcellular fractionation of the spinal cord lysates: Spinal cord tissues from Type I or Type II and their control littermates are subcellularly fractionated into cytosolic, microsomal fractions as described in Kikuchi et al. (2006) Proc Nal Acad Sci 103:6025-30. Equal amount of protein is analyzed by Western blot.

### Results

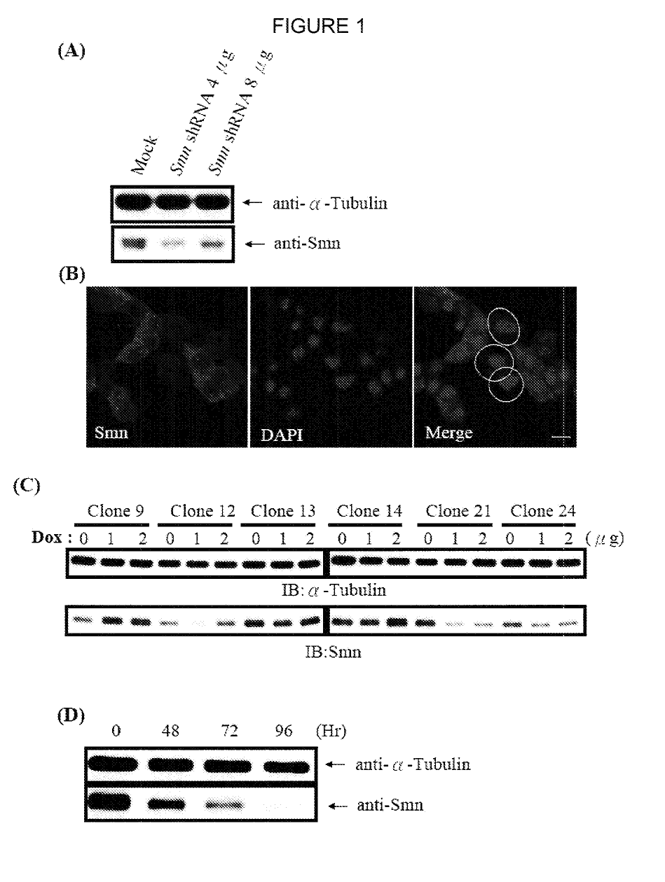

Generation of dox inducible knockdown SMN NSC34 cells: The NSC34 cell line is created by fusion of neuroblastoma cells with mouse primary motor neurons and this line had a number of motor neuron-like characteristics, including expression of Cholineacetyl-transferase and the Neurofilament triplet proteins as described in Cashman et al (1992) Dev Dyn 194:209-21. The knockdown cells can be a useful tool for studying the functions of motor neurons. The knockdown SMN NSC34 cells can be generated to precisely mimic the SMN-deficiency condition. Differentially regulated proteins in these cell lines which might contribute to SMA pathogenesis can be identified by proteomic analysis. SMN shRNA vectors are designed for knocking down SMN gene expression in NSC34 cells. NSC34 cells are transiently transfected with expressed SMN shRNA vectors, and total proteins are extracted to evaluate SMN expression by Western blot analysis. At 48 h, the SMN protein level declines in transfected NSC34 cells with SMN shRNA in contrast to non-transfected cells (FIG. 1A). This result can be confirmed by immunocytochemical analysis, which reveals less SMN expression in cytosol or nuclear gems after SMN shRNA transfection (FIG. 1B, indicated by a white circle) as compared with those non-transfected cells around them.

Recent studies have shown that cells that completely lack SMN carry out apoptotic cell death Ilangovan et al. (2003) J Biol Chem 278:30993-30999. To maintain long-term knockdown SMN expression in NSC34 cells, we used a vector system for inducible expression of shRNA can be used (see Material and Methods for details). After blasticidin resistant clonal selection, 26 single colonies are isolated, and Western blot is performed to evaluate SMN levels in the presence and absence of Dox in these clones (FIG. 1C). The SMN levels in two clones (clone21, and clone24) are successfully knocked down after Dox treatment. To compare with protein expression profiles in SMN-deficiency and control cells, the lysates with or without Dox treatment from these cell lines are further collected, are separated and are subjected to two-dimensional gel analysis.

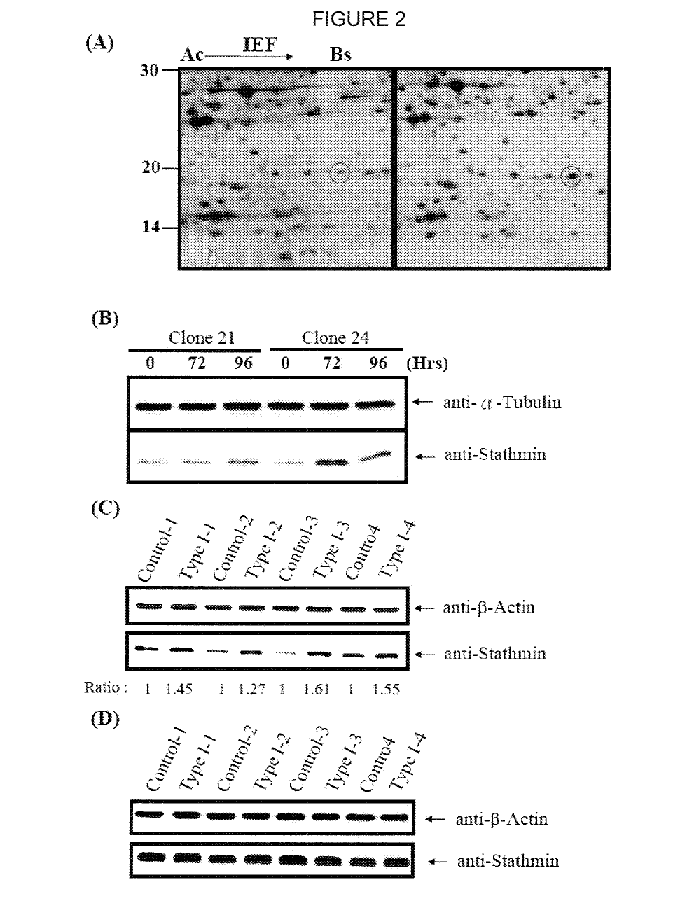

Stathmin is differentially expressed in knockdown SMN NSC34 cells and in the spinal cords of SMA-like mice. In order to compare protein expression profiles in SMN deficient and control NSC34 cells, cell lysates are extracted and are analyzed by two-dimensional analysis. A total of 25 differentially expressed protein spots are identified by MALDI-TOF mass spectrometry. In addition, to further confirm whether these differentially expressed proteins can also be found in SMA-like mice, protein samples of spinal cords from SMA-like mice and their control littermates are extracted and are analyzed using proteomic analysis. One of the spots, which is up-regulated in knockdown SMN NSC34 cells (indicated by the red circle in FIG. 2A) and in SMA-like mice, is a well-known microtubule destabilizing factor, Stathmin. To confirm the up-regulation of Stathmin, total proteins from the two stable knockdown SMN clones (clone21 and clone 24) are extracted and are analyzed for Stathmin expression by Western blot. As shown in FIG. 2B, Stahmin expression is profoundly up-regulated in both Dox-treated knockdown SMN clones in contrast to non-treated cells. Stathmin expression varied in the spinal cords of SMA-like mice is analyzed by Western blot. Spinal cord extracts from four Type I SMA-like mice and their control littermates are isolated, and are immunoblotted using an anti-Stathmin antibody. The result reveals that Stathmin expression is also up-regulated in Type I SMA-like mice (FIG. 2C). To determine whether Stathmin expression is altered only specifically in the spinal cords of SMA-like mice, Stathmin expression is analyzed in cerebellum. Cerebellum extracts are collected and are immunoblotted using an anti-Stathmin antibody, but no significant difference in levels of Stathmin expression is seen between SMA-like mice and their control littermates (FIG. 2D). This data demonstrates that Stathmin is specifically up-regulated in affected region such as the spinal cord, but not in unaffected region such as the cerebellum of SMA-like mice. This data demonstrates that Stahmin up-regulation in spinal cord is correlated with SMA.

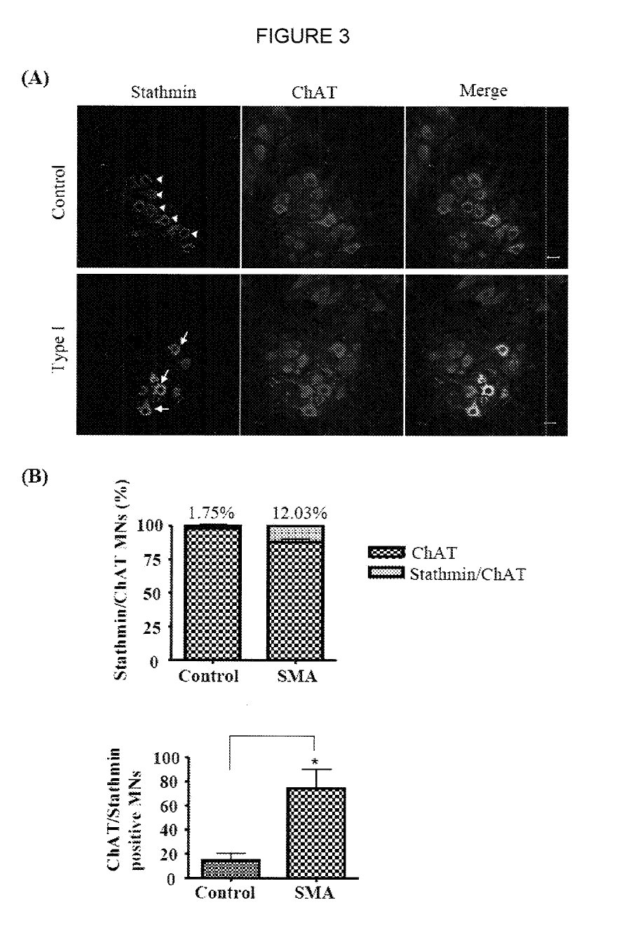

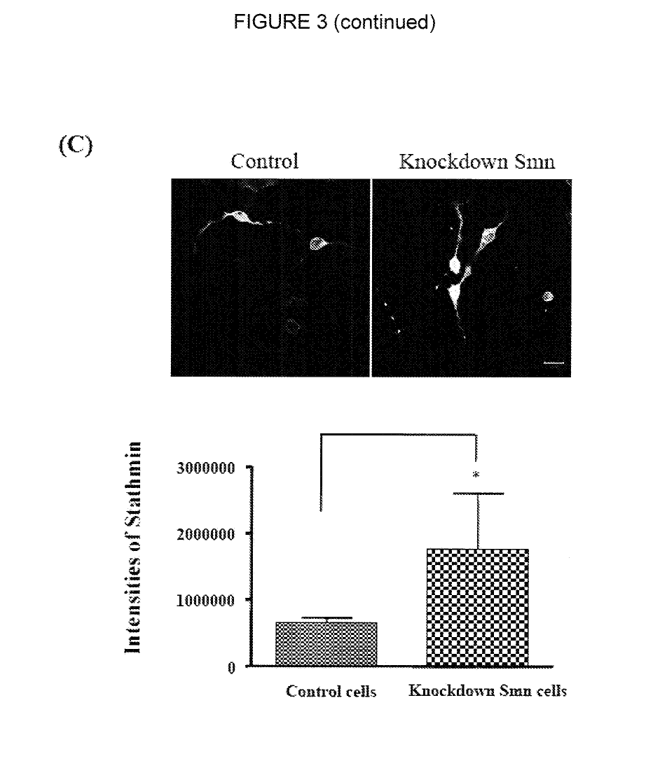

Stathmin accumulates in motor neurons in SMA mice and in knockdown SMN NSC34 cells. To determine the location of up-regulated Stathmin expression in the spinal cord, sections of spinal cords from Type I SMA-like mice and their control littermates are immunostained using the antibodies for acetylcholine transferase, a motor neuron marker, and Stathmin. Stathmin accumulation appears in motor neurons of SMA-like mice (FIG. 3, indicated by arrows), but not in control mice (FIG. 3, indicated by arrowheads). The average number of motor neurons in control and SMA-like mice is 835.5 and 617.5, respectively. The percentage of Stathmin accumulation in motor neurons is 1.7% in control mice, but increases to 12.03% in type I SMA-like mice (FIG. 3B). Quantitative analysis demonstrates that Stathmin accumulation is six times greater in SMA-like mice than in control mice (FIG. 3B) (14.5±5.6% in control littermates and 74.25±15.7% in Type I SMA-like mice).

After Dox treatment to knock down SMN expression, NSC34 cells are harvested for immunocytochemical analysis. The intensities of Stathmin in both control and knockdown SMN NSC34 cells (FIG. 3C) are quantified by MetaMorf software. The Stathmin signals increases in knockdown SMN cells in comparison with control cells (FIG. 3C). This data demonstrates that knock down SMN levels of expression correlates with the up-regulation of Stathmin.

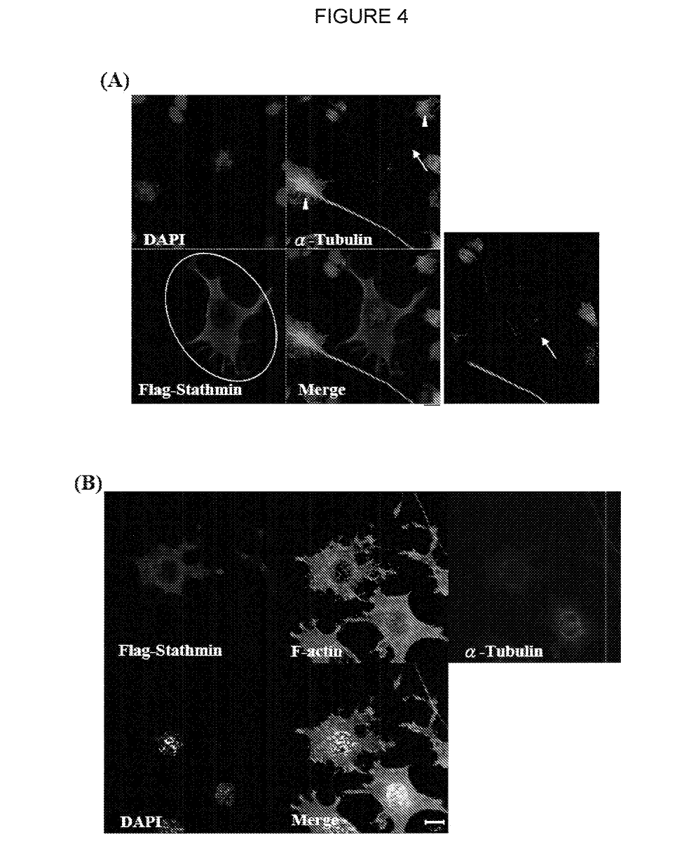

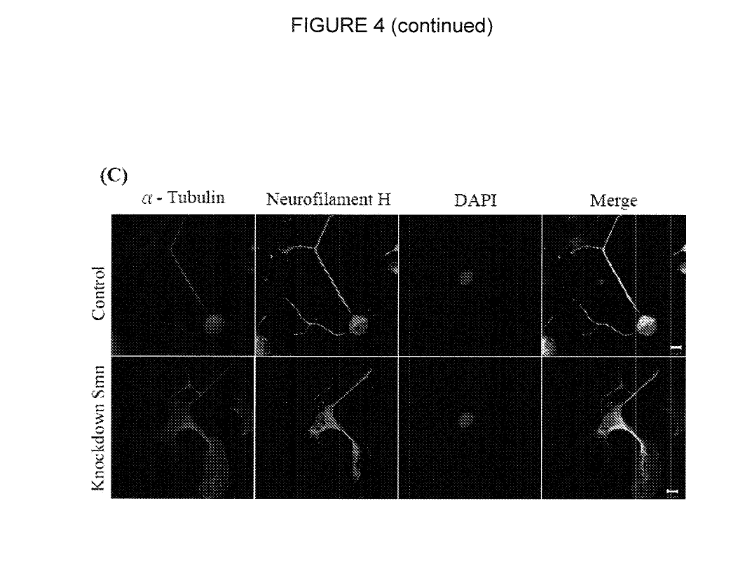

Over-expressed Stathmin disrupts microtubule organization and axon outgrowth. Microtubule destabilizing factors, such as Stathmin, can cause microtubule disorganization, and depolymerization in neuron cells, thus leading to neuronal degeneration. To test whether Stathmin up-regulation causes microtubule disassembly, Stathmin vector is introduced into NSC34 cells. The NSCB4 cell's microtubule morphology is analyzed using immunocytochemical analysis. As illustrated in FIG. 4A (and the large magnification of the white circle, indicated with an arrow in the left panel), Stathmin over-expressed in NSC34 cells reveals microtubule destructive organization. In Stathmin non-transfected normal cells, the microtubule structure is normal (indicated with arrowheads). Staining for F-actin reveals abnormal neurite branches in Stathmin transfected NSC34 cell compare with non transfected cells (FIG. 4A, B). This data demonstrates that Stathmin plays a critical role as a microtubule organizer. Further analysis with anti-α-Tubulin in knockdown SMN NSC34 cells reveals that some cells appeared discontinuous filament from the cytosl to the plasma membrane in contrast to control cells (FIG. 4C, indicated by a white circle). This data demonstrates that NSC34 cells with Stathmin over-expression show abnormal and disintegrate microtubule organization. Stahmin up-regulation is damaging to the motor neurons.

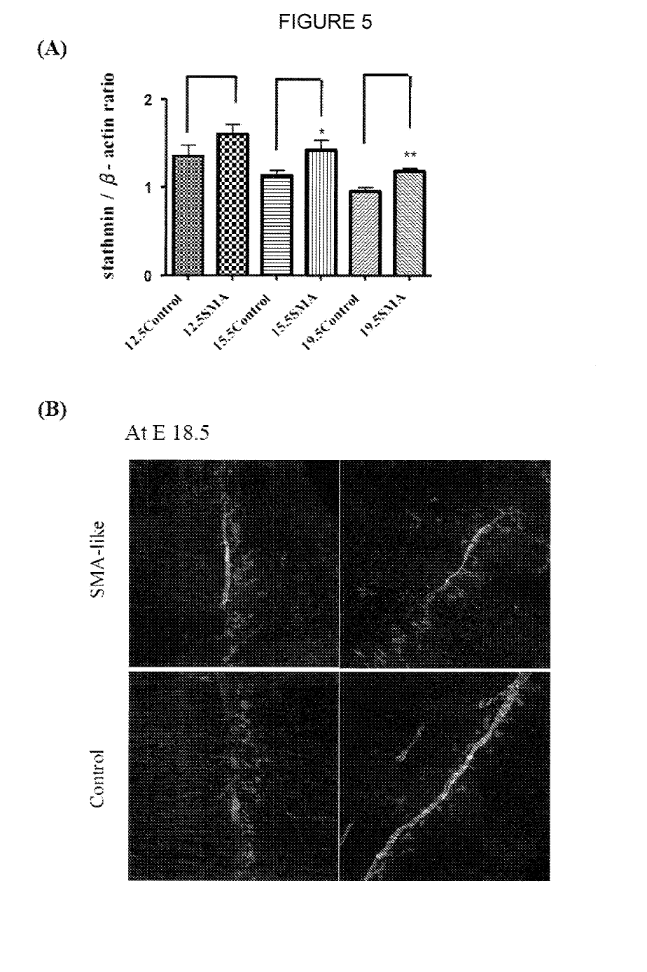

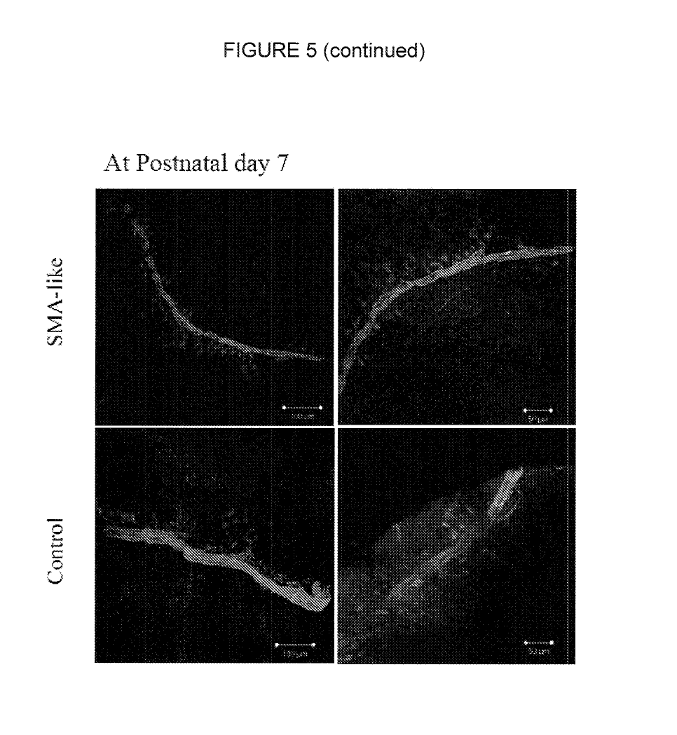

Axonopathy is demonstrated in SMA mice diaphragm muscles. Stathmin disruption in microtubule structure is detrimental to motor neurons axon outgrowth. AN investigation of defects in axon extensions of SMA-like mice is conducted. Stathmin expression of spinal cords at different embryonic stages is determined. Spinal cord extracts from SMA-like mice and their control littermates at embryonal day 12.5 (E12.5), 15.5 (E15.5) and 19.5 (E19.5) are analyzed through immunoblot by using Stathmin antibody. The results reveal no significant difference in levels of Stathmin expression at E12.5 (1.35±0.13% in control littermates and 1.6±0.1% in SMA-like embryos), however, higher levels of Stathmin expression in SMA-like mice than in control littermates at E15.5 (1.13±0.06% in control littermates and 1.4±0.11% in SMA-like embryos) and E19.5 (0.95±0.05% in control littermates and 1.2±0.03% in SMA-like embryos) are observed (FIG. 5A). Whole-mount staining of diaphragm muscles with an antibody against Neurofilament is performed to analyze axonal projection in peripheral nerve in control and Type I SMA-like mice at E18.5 and postnatal day 7. An investigation of the distribution of synapses can employ labeled AChR clusters with Texas red-conjugated α-bungarotoxin. As illustrated in FIG. 5B, axonal projections are present in the diaphragm muscles in both control and SMA-like mice, but axons in SMA-like mice are profoundly thinner than in control littermates both at E18.5 and postnatal day 7. Both control and SMA-like mice can form neuromuscular junctions in which the nerve terminals colocalized with AChR clusters (FIG. 5B). Changes in axonal projections in SMA-like mice can deteriorate functional muscles and can further lead to respiratory failure which is the cause of death in most SMA patients and animal models. The up-regulation of Stahmin in SMA-like mice damages microtubule maintenance during axon outgrowth, causing axon degeneration, and contributing to axonopathy found in the diaphragm muscles of SMA-like mice.

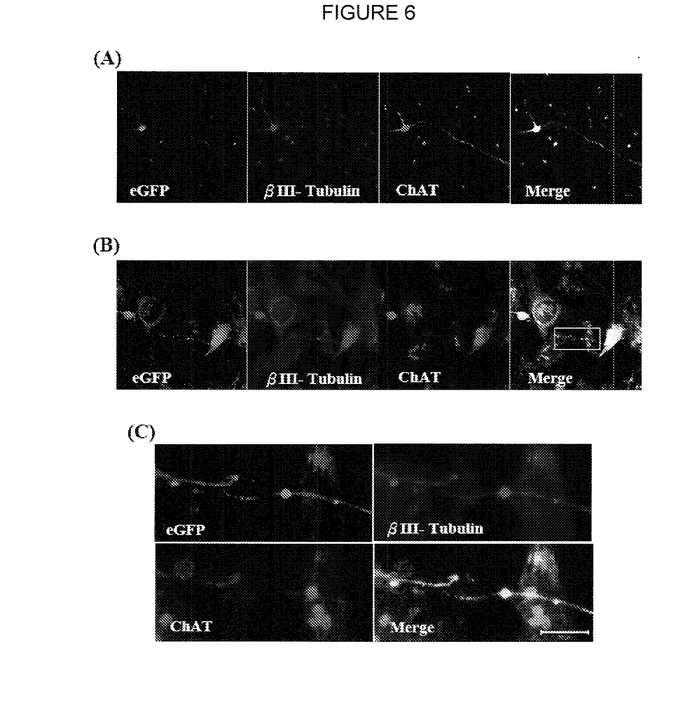

Stathmin over-expression causes motor neuron axon degeneration and cell death. An expressed-Stathmin vector is introduced into primary culture motor neurons. Expressed-eGFP vector is used as a control. The eGFP transfected motor neurons have normally extended long and intact axonal process (FIG. 6A). The Stathmin transfected motor neurons, however, axons remained extended long distances but the axon is fragmented (FIG. 6B, C). This data demonstrates that over expression of Stathmin causes motor neuron axon degeneration and cell death.

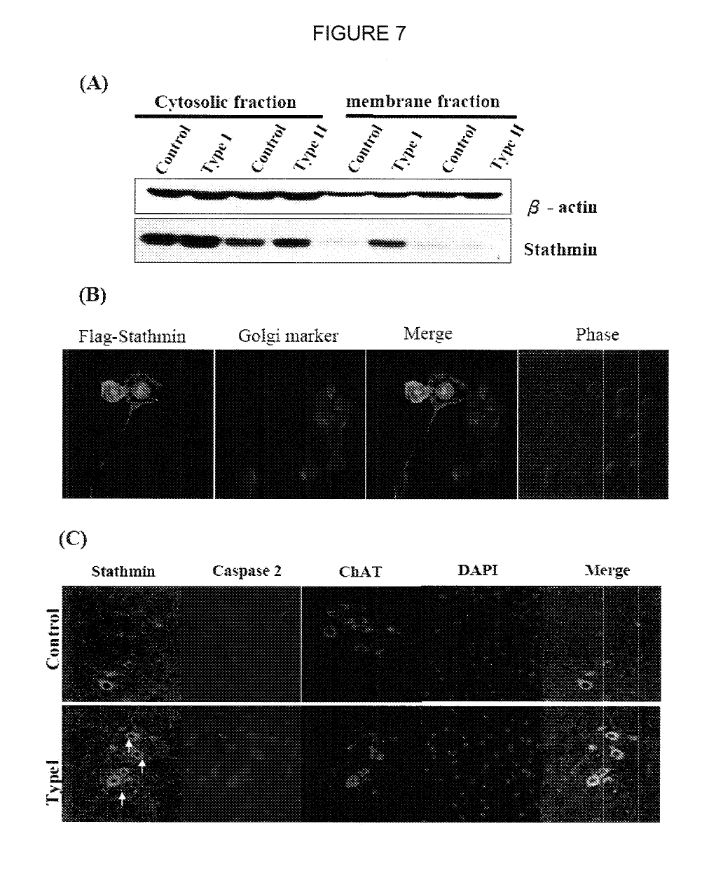

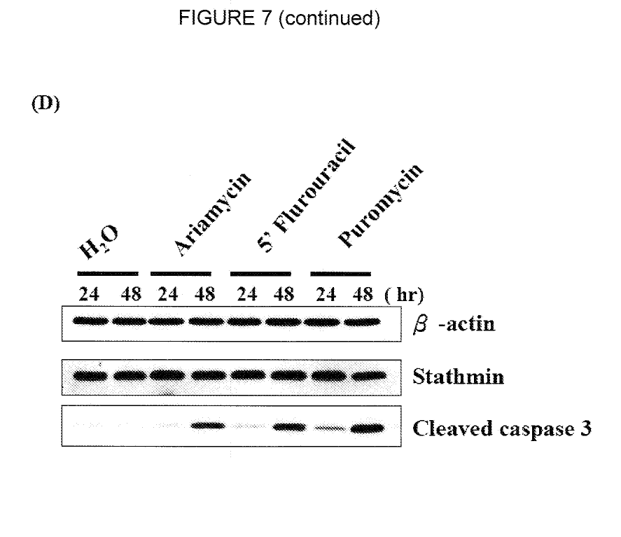

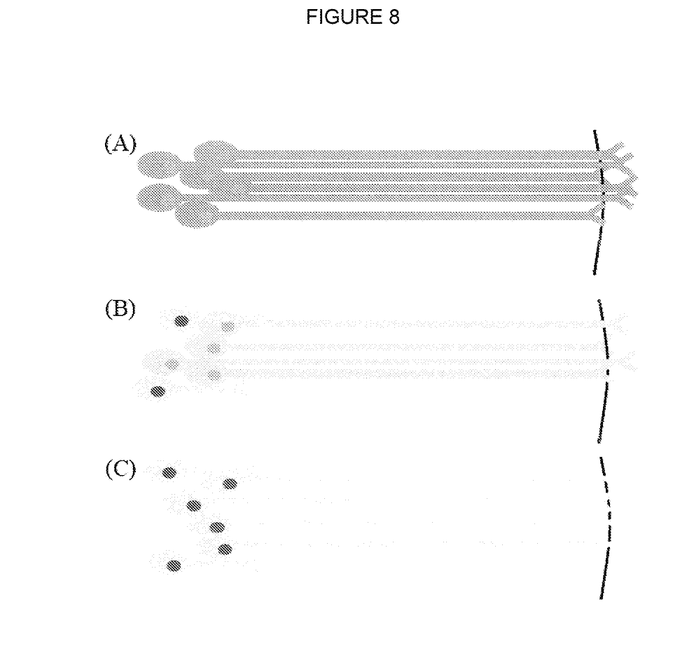

Stathmin accumulation is harmful to motor neurons. As previously illustrated in FIG. 3A, rare perinuclear localizations of Stathmin can be observed in a section of spinal cord form a control mouse. This perinuclear localization is consistent with the Golgi apparatus (Strey et al. (2004) Am J. Pathol 165:1701-18). Stathmin accumulates in the cytoplasm in SMA-like mice. The accumulated Stathmin can be associated with the golgi apparatus. Subcellular fractionation of spinal cord lystaes from SMA-like mice and control littermates are isolated and are analyzed for Stathmin expression by Western blot. The result reveals that the Stathmin expression in Type I SMA-like mice increases not only in cytosolic fractions but also in microsome fractions (FIG. 7A). Fragmented golgi apparatus are found in Stathmin-overexpressing cells (FIG. 7B). Sections of spinal cords from Type I SMA-like mice and control littermates are immunostained using the antibodies for caspase 2, an indicator of damaged Golgi. Triple labeling with ChAT, Stathmin and caspase 2 reveals that the majority of the caspase 2-positive cells are accumulated Stathmin motor neurons (FIG. 7C, indicated with arrows). Stathmin expression in NSC34 cells is analyzed by treating several apoptosis-inducing drugs through Western blot. Stathmin expression level in NSC34 cells is not significantly influenced by treating those apoptosis-inducing drugs. This data demonstrates that Stathmin accumulation contributes to the motor neuron death in SMA.

Embodiments and the examples described herein are exemplary and not intended to be limiting in describing the full scope of compositions and methods of these disclosure. Equivalent changes, modifications and variations of some embodiments, materials, compositions and methods can be made within the scope of the present disclosure, with substantially similar results.

What is claimed is:

1. A recombinant genetic vector comprising at least one copy of a Stathmin inhibitor, the Stathmin inhibitor encoding an antisense nucleic acid targeting Stathmin RNA, wherein the target sequence of the antisense nucleic acid comprises CGT TTG CGA GAG AAG GAT A (SEQ ID. NO. 6).

2. The recombinant genetic vector of claim 1, wherein the recombinant genetic vector is a viral vector.

3. The recombinant genetic vector of claim 2, wherein the viral vector is an adenovirus, adeno-associated virus, herpes virus, or lentivirus.

4. The recombinant genetic vector of claim 3, wherein the virus is non-virulent.

5. A recombinant genetic vector comprising at least one copy of a Stathmin inhibitor, the Stathmin inhibitor encoding a nucleic acid selected from the group consisting of an siRNA, an shRNA, and an anti-Stathmin ribozyme that targets Stathmin RNA, wherein the target sequence of the RNAi nucleic acid comprises CGT TTG CGA GAG AAG GAT A (SEQ ID. NO. 6).

6. The recombinant genetic vector of claim 5, wherein the recombinant genetic vector is a viral vector.

7. The recombinant genetic vector of claim 6, wherein the viral vector is an adenovirus, adeno-associated virus, herpes virus, or lentivirus.

8. The recombinant genetic vector of claim 7, wherein the virus is non-virulent.

9. The recombinant genetic vector of claim 5, wherein the shRNA-encoding sequence is operably attached to a U6 promoter.

10. A Stathmin RNA-targeted nucleic acid comprising a Stathmin targeted siRNA or a Stathmin targeted morpholino oligo, wherein the target sequence of the Stathmin targeted siRNA or a Stathmin targeted morpholino oligo comprises CGT TTG CGA GAG AAG GAT A (SEQ ID. NO. 6).


SEQUENCE LISTING (6 sequences)

SEQ ID NO: 1 - DNA, 6762 bp, Homo sapiens
  misc_feature (1)..(6762): Genomic DNA sequence of the stathmin STMN1 gene (from Genbank Accession No. NC_000001.9..gi89161185)
aggggcactgctctgtccgagtgctgcccttggggcgaggcgggcatgtggctctacaaggtggagtccaggcggccaaagtttggaaaggtagggaaggacccccccgccctccgcctgctccgccctgcccttgttctcgagaatggggagctggttcggacctagtccggggtccactgccacgccctcttccacggcgagaccacccccctagtcccaggcccacacctggggatgctccccaggcgccctgcaacccccggcattgtcctcctgccctccgggacaggactacacttcccgaggtgcttcggggtcccggggggcggcgctccacgcgggttgtggggggcgggggcggcacgtgccgccgctctcggccaatgcggagccccgcgcggaggtcacgtgcctctgtttggcgcttttgtgcgcgcccgggtctgttggtgctcagagtgtggtcaggcggctcggactgagcaggtgggtgcggggctcggaggaggcggcggctggctgaggccagcaagagggacgcggtcggcgggaggggctgggccgtggcagcgaccccctgctgcagggcggcgggcggggctgcgggcctcggaggggttggtgggcgggggtcgctccgctttgtgtgtggctcgggcggagcctcgcctttgtccccgctctccgggggcgcggctgttcgtgggcagggggctgggcgatcaccgggcgtccgctccggggtgccgtcgaggagacaatagggggcgtgggccctcgtttacctccctccctccctcccttccctgcgggccccgccgggttccccattgtctgaagggacggggcggtgccccagggaccagcggctttaggaccaaactgcgggcagccagggccgcgaccctccctgcgaccgtcccctggcgaccgcagctggtgattgaggggcggcgctcccgggccccacgagggttcttctgtcttcgcggccggacgcgcggacagcgtgggtggcggcaggttgggcatggggacggcgggaggcggtggcgagctcaccgcgggaccacccgggggcctgttcccggggcctgccccacccgctgaactgtgaagggggtggtggcggcggcctggaggtgtttttggcgggagttggggggggcgtccgcgcagggggagtcaggcaggggcggagttacccggattggaccgttagccccgcccacccctccccttcccacgcgcgcgggctccggggtgttgagttcggggagattcgaaaaggcgcggggaggaagggggcggggccaggggccggagcgcaaggcgtgctctgattggccgggggcgaccggtcctcttttcctcgcccggaccagggccacgcccatcctgggtccggtgctgcgtctaattctctgcttttcttaaatcttgtcgctgcctctgattttaattcctagcttttgggaacctgtcatcctacgtttttggtactagctggcgtctacaaaagtcataatgttaaaaagatcaacaagagatacagcatttttcatgacataacggcagcaaatataagtcaaaatctagaggttcataaacattttgcttgctgttgggcaaggaagcttaaacctgagggacaataggagttcaacattattggttactattagcttgggcgttttcttatccaccacgtcagacacagacaaagcaggggtgggtatttcatttgcacaatgagttgtaggcagtattaagatggctccgggggcactgttgagttgaatctggaatatcttcttacagtttcggtgaaatgttaaagagtttatgggggaaaaattcttcacccttgtgactttgtctgattttaaaaatccaagagttttatgaccgagaaagctcagttaacttgattttctggaaccaatatcatattcaggtcatatttcccaatgtttatttagtagattttgataatttttttcgtggttaatttagacgtctttattccacgtatttttctgacgatgtatgtagatgtgatgtgagatttttttgggttgatgacatatagaaaggcaaagaaagtgattgcatgtttttgaaaatcattttcaggactttccttatcccagttgattgtgcagaatacactgcctgtcgcttgtcttctattcaccatggcttcttctggtaggtaatctatttggaaaatctgaaattgtaatgggcttatgattttagattgagatggctcaggtcttcgcctttgatttggcacttatgttttggtcttaccaaaacctattttatgaataggagaagaatttaaaaatgattatcacttgaatgtgccgagagctcgtaattgtttattggacagtttggcttagtctgaagcaaaattgtggagtttgcacaagtcttttgtttatgaaagcgattgtcagatactgatgtctcaaaacagtatttattaatccaaaaatgttgagctttgtttttctgggagatggtttttattttttttgagacagggtctcaccttgttgcccaggctggagtgcagtggcttaattatagctcattgcagccttgacttctggagctcaagtgggctcaagcgattctcccacctcagcctccatagcatctgggactatcgacatgggccaccacacccacctaatcaaaaaaaattttttttgtagagatgggctttccctttgttgcccaggctggtctcaaacttcagggctcaagggatcttcccatgttggcctcccacggtgctgggattataggcatgagccatggtacctggccttgggaaatggtatttagataataatatcttgcctgcaaatacatcttcccctagtgtcagtagactgataggaataaaaaggggaaaaaaaacacaactttcctcatcagccctagtttaatacattaaattgatttgggttttagaaaattatagtacagtttattagaacaggagaatcctggttttctgaattataaatataatcaattctagatatccaggtgaaagaactggagaagcgtgcctcaggccaggcttttgagctgattctcagccctcggtcaaaagaatctgttccagaattccccctttcccctccaaagaagaaggatctttccctggaggaaattcagaagaaattagaagctgcagaagaaagacgcaaggtaaacgaagcaattcacagaaagcaggatattaatttatgtaatgggcagatcaattttatttctataacaggaagaaaacagaattgtagctacactgtgattattacatatgccagtgactggaaggaaataccagtcctcatttattgaactcctgttacatgcccgctcctttgtttatatttttctcctttaatacatggtgttgcctcaaataatggaaattagaaacagtttcaggaatgttaagtcgtttttctgcagtcatacaactagtaagtgttggggtcagaattcaagccctggtctatcttaaaccaaagctcatgcttctctcatgcttcctctttgaaaagatttgttgccagatgattctttggcactttggttttgttttttgagagctgtacaataacattttaaattgctagtgtgattgtgttgctcagctggtatcatggtagcttttctcttatctaaacaattctattataaagtaactatctttaaaagctaatcagaagaatcaataaatattaatatgctagttgtagaaaatttgggaaatacagaaatttataaatggaaattaaaagtattcattatcccgcttccgagaagcaaccagtgttaacattttggtgtgtttctttccattcaatgttactcattaacaactgtacataacttttcatttaactccctttctcaacaaccctgataggattgattttaaagctggggcaagtgaggcacaaaaggtaaggtaataaccttccccagaccagcatagtgatttgtagtatacacacacacccgcccgaagagcctcagtgcttacactaaggagtcgtcttctatgcaatagtgtgagttcatggagtaggaggaagcaatacaaccaaaggttggacagtggaaagctttttagacatcaaccctggccctgtagtcattagcctgtgctttacatagtaactggctaaatataatgaaactcccatcatgactaggatttggcagaagagaatcaatagaaccagtgtcagatgctctgtggttatcctgcaagtcagtggttccaatgtgttttgagaacaagctgttctgttgaaggggtcataccaaggtatggtctggtaattaatgcagtttcctgagacaaaagctaataagccttttccttgaaacaaatttttctgtcttaaatagtaatctacagacttagtcttgaatttcctatcattgttttatcagttatggttaaaatttttacaatgagctagttttctttgggtagcttttgaagttaaatagtgaaattctttacaataaaagtgccactcgcaagtacatattcctcaagtcatccagataccattaagcagtaaatcttacaaggatttccttaaggactaattgggtaagatttctgaacagataagcacttttccaaagttaaatacaaatactagaaaaagaatatcattttcacagtattttatgatagggataattcaggtcctaattttggtgttatttaaagggaccttattttctgccccttttcaatccccttagtaaattatttttattgtaattttaacttatgctgaatacattttattttttgaggcagagtctctgtcacccagggtggagtgcagtggcacgatctcagttcactgcaacctccacctcccgggttcaagcaattcttctgcctcagcctcccaagtaacttagactacaggcacccgccaccacgcccggctaatttttgtatttttagtataggtaggatttcaccatgttggccaggctggtcttgaactcctgacctcaaatgatccacccaccttggcctcccaaagtgctgggattacaggcgtgagccaccacacccagcctgaatacattttagagtgcctagcctattaaacttttttttccagtcccatgaagctgaggtcttgaagcagctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaaggttggtttcttactttgtaaaagggttgagcttggagtttgatgcaccaatgagttggcttgaactaagtgctttgataaaaggtgtttggtgtctttttgtcatccattttggggcttaacatattaaatgaaaggtatattttaagatggaatattcagtaattcccagcataattgcacagtccttggaagtccagtaggcagcttgttaggttctacaagggacccaggagatttgatgatgatgtctcagaacttaaattgtgtggttcccacaggctgtaatatatgcactgaggttgtgttgggcctctttgaggtgggggctgggggtcgtgacttgacaggcttttttttttttttttttttttgactgatgacaccttacccttcctttacaggataagcacattgaagaagtgcggaagaacaaagaatccaaagaccctgctgacgagactgaagctgactaatttgttctgagaactgactttctccccatccccttcctaaatatccaaagactgtactggccagtgtcattttattttttccctcctgacaaatattttagaagctaatgtaggactgtataggtagatccagatccagactgtaagatgttgttttaggggctaaaggggagaaactgaaagtgttttactctttttctaaagtgttggtctttctaatgtagctatttttcttgttgcatcttttctacttcagtacacttggtgtactgggttaatggctagtactgtattggctctgtgaaaacatatttgtgaaaagagtatgtagtggcttcttttgaactgttagatgctgaatatctgttcacttttcaatcccaattctgtcccaatcttaccagatgctactggacttgaatggttaataaaactgcacagtgctgttggtggcagtgacttcttttgagttaggttaataaatcaagccatagagcccctcctggttgatacttgttccagatggggcctttggggctggtagaaatacccaacgcacaaatgaccgcacgttctctgccccgtttcttgccccagtgtggtttgcattgtctccttccacaatgactgctttgtttggatgcctcagcccaggtcagctgttactttctttcagatgtttatttgcaaacaaccattttttgttctgtgtcccttttaaaaggcagattaaaagcacaagcgtgtttctagagaacagttgagagagaatctcaagattctacttggtggtttgcttgctctacgttacaggtggggcatgtcctcatcctttcctgccataaaagctatgacacgagaatcagaatattaataaaactttatgtactgctgtagcaa

SEQ ID NO: 2 - DNA, 1730 bp, Homo sapiens
  misc_feature (1)..(1730): cDNA of Stathmin STMN1 mRNA (longest transcript variant from Genbank Accession No. NM_203401.1.. gi44890051; also see variants 2 and 3 thereof)
atcaccgggcgtccgctccggggtgccgtcgaggagacaatagggggcgtgggccctcgtttacctccctccctccctcccttccctgcgggccccgccgggttccccattgtctgaagggacggggcggtgccccagggaccagcggctttaggaccaaactgcgggcagccagggccgcgaccctccctgcgaccgtcccctggcgaccgcagctggtgattgaggggcggcgctcccgggccccacgagggttcttctgtcttcgcggccggacgcgcggacagcgtgggtggcggcaggactttccttatcccagttgattgtgcagaatacactgcctgtcgcttgtcttctattcaccatggcttcttctgatatccaggtgaaagaactggagaagcgtgcctcaggccaggcttttgagctgattctcagccctcggtcaaaagaatctgttccagaattccccctttcccctccaaagaagaaggatctttccctggaggaaattcagaagaaattagaagctgcagaagaaagacgcaagtcccatgaagctgaggtcttgaagcagctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaaggataagcacattgaagaagtgcggaagaacaaagaatccaaagaccctgctgacgagactgaagctgactaatttgttctgagaactgactttctccccatccccttcctaaatatccaaagactgtactggccagtgtcattttattttttccctcctgacaaatattttagaagctaatgtaggactgtataggtagatccagatccagactgtaagatgttgttttaggggctaaaggggagaaactgaaagtgttttactctttttctaaagtgttggtctttctaatgtagctatttttcttgttgcatcttttctacttcagtacacttggtgtactgggttaatggctagtactgtattggctctgtgaaaacatatttgtgaaaagagtatgtagtggcttcttttgaactgttagatgctgaatatctgttcacttttcaatcccaattctgtcccaatcttaccagatgctactggacttgaatggttaataaaactgcacagtgctgttggtggcagtgacttcttttgagttaggttaataaatcaagccatagagcccctcctggttgatacttgttccagatggggcctttggggctggtagaaatacccaacgcacaaatgaccgcacgttctctgccccgtttcttgccccagtgtggtttgcattgtctccttccacaatgactgctttgtttggatgcctcagcccaggtcagctgttactttctttcagatgtttatttgcaaacaaccattttttgttctgtgtcccttttaaaaggcagattaaaagcacaagcgtgtttctagagaacagttgagagagaatctcaagattctacttggtggtttgcttgctctacgttacaggtggggcatgtcctcatcctttcctgccataaaagctatgacacgagaatcagaatattaataaaactttatgtactgctgtagcaaaaaaaaaaaaaaaaaa

SEQ ID NO: 3 - protein, 149 aa, Homo sapiens
MASSDIQVKELEKRASGQAFELILSPRSKESVPEFPLSPPKKKDLSLEEIQKKLEAAEERRKSHEAEVLKQLAEKREHEKEVLQKAIEENNNFSKMAEEKLTHKMEANKENREAQMAAKLERLREKDKHIEEVRKNKESKDPADETEAD

SEQ ID NO: 4 - DNA, 59 bp, Artificial
  chemically synthesized oligonucleotide
gatcccgtagctaatagtacagaacttcaagagagttctgtactattagctactttttt

SEQ ID NO: 5 - DNA, 58 bp, Artificial
  chemically synthesized oligonucleotide
agcttaaaaaagtagctaatagtacagaactctcttgaagttctgtactattagccat

SEQ ID NO: 6 - DNA, 19 bp, Artificial
  chemically synthesized oligonucleotide
cgtttgcgagagaaggata